(19) United States
(12) Patent Application Publication
(10) Pub. No.: US 2023/0313112 A1
(43) Pub. Date: Oct. 5, 2023

(54) Uniform Material Distribution Control Method and System for Fermentation Tank

(71) Applicants: BEIJING GREENTECH SCIENCE AND TECHNOLOGY CO., LTD., Beijing (CN); BEIJING ZHONGKE BOLIAN INTELLIGENT TECHNOLOGY CO., LTD., Beijing (CN)

(72) Inventors: Xue Huang, Beijing (CN); Tongbin Chen, Beijing (CN); Guofei Li, Beijing (CN); Chaowen Huang, Beijing (CN); Xunkuo Zhu, Beijing (CN); Jingwei Zhang, Beijing (CN)

(21) Appl. No.: 18/127,638

(22) Filed: Mar. 28, 2023

(30) Foreign Application Priority Data
Mar. 30, 2022 (CN) 202210326433.0

(51) Int. Cl. C12M 1/34; C12M 1/02

(52) U.S. Cl. CPC C12M 41/42; C12M 27/00

(57) ABSTRACT

The application discloses a uniform material distribution control method and system for fermentation tank in an aerobic fermentation apparatus for organic solid waste. A total amount of materials in the fermentation tank and an average material distribution amount at each material distribution point are acquired; a target speed for material distribution at each material distribution point is determined according to the total amount of materials, the target speed is corrected according to the average material distribution amount and a target average material distribution amount when the average material distribution amount is not equal to the target average material distribution amount, and an advanced announcement time of the target speed is calculated according to the corrected target speed, a distance in the fermentation tank and a closed-loop regulation lag time; a closed-loop control to the total amount of materials is performed based on the advanced announcement time of the target speed.

### CROSS REFERENCE TO RELATED APPLICATIONS

The present application claims the benefit of Chinese Patent Application No. 202210326433.0 filed on Mar. 30, 2022, the contents of which are incorporated herein by reference in their entirety.

### FIELD OF THE PRESENT INVENTION

The present invention relates to the technical field of aerobic fermentation treatment for organic solid wastes, in particular to a uniform material distribution control method and system for fermentation tank.

### BACKGROUND OF THE PRESENT INVENTION

Organic solid wastes are involved in various aspects of national production and living. All of domestic wastes generated in daily life, sludges left after domestic sewage treatment, medical wastes generated in the medical industry, and residues from recycled paper mills are regarded as organic solid wastes. The organic solid wastes have a complex composition, high organic substance and moisture content, are easy to decay, and have a foul smell; and they are rich in a large number of parasitic eggs, pathogens such as bacteria and viruses, and organic pollutants such as antibiotics, such that they are very prone to cause diffusion of pollution sources, spread diseases, and pollute water sources, soil and air, thereby seriously threatening human health and environmental safety.

In an existing aerobic fermentation process for organic solid wastes, a long fermentation tank is usually used for material distribution control, and a multi-point fixed-point material distribution method is specifically adopted. In the multi-point fixed-point material distribution method, the fermentation tank is divided into multiple material distribution points, and an equal amount of sludge is distributed at each material distribution point, so as to achieve approximately uniform material distribution for the fermentation tank.

However, in the case of the multi-point fixed-point material distribution method, a uniform material distribution cannot be achieved, and a sludge pile will be formed at each material distribution point, thereby affecting aeration uniformity in a later fermentation process.

### SUMMARY OF THE PRESENT INVENTION

Based on this, an embodiment of the present application provides a uniform material distribution control method and system for fermentation tank, capable of solving the problem of non-uniform material distribution in an aerobic fermentation apparatus for organic solid wastes during the fermentation process.

In a first aspect, a uniform material distribution control method for fermentation tank is provided, which comprises:

acquiring a total amount of materials in the fermentation tank and an average material distribution amount at each material distribution point;

determining a target speed for material distribution at each material distribution point according to the total amount of materials, wherein the target speed is corrected according to the average material distribution amount and a target average material distribution amount when the average material distribution amount is not equal to the target average material distribution amount, and an advanced announcement time of the target speed is calculated according to the corrected target speed, a distance in the fermentation tank and a closed-loop regulation lag time; and

performing a closed-loop control to the total amount of materials based on the advanced announcement time of the target speed.

Optionally, acquiring an average material distribution amount at each material distribution point comprises:

acquiring a cumulative material distribution amount in the fermentation tank, and determining the average material distribution amount at each material distribution point according to a number of material distribution points in the fermentation tank.

Optionally, after determining the target speed for material distribution at each material distribution point according to the total amount of materials, the method further comprises:

calculating the advanced announcement time of the target speed according to the target speed, the distance in the fermentation tank and the closed-loop regulation lag time when the average material distribution amount is equal to the target average material distribution amount; and

performing a closed-loop control to the total amount of materials based on the advanced announcement time of the target speed.

Optionally, calculating the advanced announcement time of the target speed according to the target speed, the distance in the fermentation tank and the closed-loop regulation lag time comprises:

determining the regulation lag time Δt and the target speed vₜ according to a first formula which is specifically as follows:

$$Δ t = {k}_{t} \frac{l \frac{{w}_{0}}{w} - s}{{v}_{0}} , {v}_{t} = \frac{l}{\frac{w}{{f}_{i}} - Δ t}$$

where, kₜ represents a compensation coefficient, s represents a current position, l represents a total length, w represents a target material distribution amount, w₀ represents a current material distribution amount, v₀ represents a current speed, and fₜ represents an instantaneous material flow rate.

Optionally, performing a closed-loop control to the total amount of materials based on the advanced announcement time of the target speed comprises:

regulating, by PID, the total amount of materials in the fermentation tank;

acquiring a current feedback speed according to an output set frequency of current PID regulation; and

regulating, by PID, the current total amount of materials based on the current feedback speed.

Optionally, an output calculation result of PID regulation is acquired according to a second formula which is specifically as follows:

$$y = {K}_{p} [ ( b \cdot {v}_{t} - {v}_{0} ) + \frac{1}{{T}_{I} \cdot s} ( {v}_{t} - {v}_{0} ) + \frac{{T}_{D} \cdot s}{a \cdot {T}_{D} \cdot s + 1} ( c \cdot {v}_{t} - {v}_{0} ) ]$$

where, y represents the output calculation result of PID regulation, Kₚ represents a proportional gain, s represents a Laplace operator, b represents a proportional action weight, vₜ represents a target speed, v₀ represents a current speed, T_{I} represents an integral action time, T_{D} represents a differential action time, a represents a differential delay coefficient, and c represents a differential action weight.

In a second aspect, a uniform material distribution control system for fermentation tank is provided, which comprises:

an acquisition module, configured to acquire a total amount of materials in the fermentation tank and an average material distribution amount at each material distribution point;

a calculation module, configured to determine a target speed for material distribution at each material distribution point according to the total amount of materials, correct the target speed according to the average material distribution amount and a target average material distribution amount when the average material distribution amount is not equal to the target average material distribution amount, and calculate an advanced announcement time of the target speed according to the corrected target speed, a distance in the fermentation tank and a closed-loop regulation lag time; and

a control module, configured to perform a closed-loop control to material distribution based on the advanced announcement time of the target speed.

Optionally, in the acquisition module, acquiring an average material distribution amount at each material distribution point comprises:

acquiring a cumulative material distribution amount in the fermentation tank, and determining the average material distribution amount at each material distribution point according to a number of material distribution points in the fermentation tank.

Optionally, after the target speed for material distribution at each material distribution point is determined according to the total amount of materials, the calculation module is further configured to calculate the advanced announcement time of the target speed according to the target speed, the distance in the fermentation tank and the closed-loop regulation lag time when the average material distribution amount is equal to the target average material distribution amount; and

the control module is further configured to perform a closed-loop control to the total amount of materials based on the advanced announcement time of the target speed.

Optionally, in the control module, performing a closed-loop control to the total amount of materials based on the advanced announcement time of the target speed comprises:

regulating, by PID, the total amount of materials in the fermentation tank;

acquiring a current feedback speed according to an output set frequency of current PID regulation; and

regulating, by PID, the current total amount of materials based on the current feedback speed.

In a technical solution provided by the embodiment of the present application, firstly, a total amount of materials in the fermentation tank and an average material distribution amount at each material distribution point are acquired; secondly, a target speed for material distribution at each material distribution point is determined according to the total amount of materials, wherein, the target speed is corrected according to the average material distribution amount and a target average material distribution amount when the average material distribution amount is not equal to the target average material distribution amount, and an advanced announcement time of the target speed is calculated according to the corrected target speed, a distance in the fermentation tank and a closed-loop regulation lag time; and finally, a closed-loop control to the total amount of materials is performed based on the advanced announcement time of the target speed. In the present application, a PID closed-loop regulation is used to prompt the apparatus to continuously distribute materials according to the target speed, so as to achieve the purpose of continuous distribution, while the advanced announcement of the target speed offsets the hysteresis of the closed-loop regulation. During the material distribution process, the target speed is continuously corrected according to the total amount of materials and the average material distribution amount, so as to achieve the purpose of uniform material distribution.

### BRIEF DESCRIPTION OF THE DRAWINGS

Hereafter, in order to more clearly illustrate the implementations of the present invention or the technical solutions in the prior art, drawings required in the description of the implementations or the prior art will be briefly introduced. It is obvious that the drawings in the following description are only illustrative, and for those skilled in the art, other implementation drawings may also be obtained by derivation according to the provided drawings without creative efforts.

FIG. 1 is a flow chart of steps of a uniform material distribution control method for fermentation tank provided in an embodiment of the present application;

FIG. 2 is a flow chart of a uniform material distribution control method for fermentation tank provided in an embodiment of the present application; and

FIG. 3 is a block diagram of a uniform material distribution control system for fermentation tank provided in an embodiment of the present application.

### DETAILED DESCRIPTION OF THE EMBODIMENTS

Hereafter, the implementations of the present invention will be illustrated by means of specific embodiments, and other advantages and effects of the present invention will become apparent to those skilled in the art from the contents disclosed in the description. Obviously, the described embodiments only represent some embodiments of the present invention, but are not exhaustive. Based on the described embodiments of the present disclosure, all the other embodiments that may be obtained by one of ordinary skill in the art without creative efforts will fall into the scope of protection of the present invention.

In order to facilitate the understanding of the embodiments, firstly, a uniform material distribution control method for fermentation tank disclosed in an embodiment of the present application is introduced in detail.

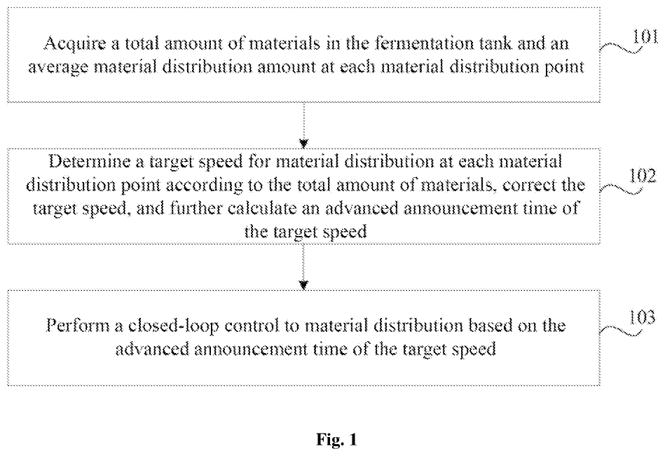

Referring to FIG. 1, a flow chart of steps of a uniform material distribution control method for fermentation tank provided in an embodiment of the present application is shown. The method may be applied to an aerobic fermentation apparatus for organic solid wastes, and may comprise the following steps:

At step 101, a total amount of materials in the fermentation tank and an average material distribution amount at each material distribution point are acquired.

In an embodiment of the present application, acquiring an average material distribution amount at each material distribution point by using a weighing measurement device comprises: acquiring a cumulative material distribution amount in the fermentation tank, and determining the average material distribution amount at each material distribution point according to a number of material distribution points in the fermentation tank.

At step 102, a target speed for material distribution at each material distribution point is determined according to the total amount of materials, wherein the target speed is corrected according to the average material distribution amount and a target average material distribution amount when the average material distribution amount is not equal to the target average material distribution amount, and an advanced announcement time of the target speed is calculated according to the corrected target speed, a distance in the fermentation tank and a closed-loop regulation lag time.

Specifically, in an embodiment of the present application, the regulation lag time Δt and the target speed vₜ is determined according to a first formula which is specifically as follows:

$$Δ t = {k}_{t} \frac{l \frac{{w}_{0}}{w} - s}{{v}_{0}} , {v}_{t} = \frac{l}{\frac{w}{{f}_{t}} - Δ t} \qquad ( 1 )$$

where, kₜ represents a compensation coefficient, s represents a current position, l represents a total length, w represents a target material distribution amount, w₀ represents a current material distribution amount, v₀ represents a current speed, and fₜ represents an instantaneous material flow rate.

At step 103, a closed-loop control to the total amount of materials is performed based on the advanced announcement time of the target speed.

In an embodiment of the present application, the step 103 specifically comprises:

regulating, by PID, the total amount of materials in the fermentation tank;

acquiring a current feedback speed according to an output set frequency of current PID regulation; and

regulating, by PID, the current total amount of materials based on the current feedback speed.

Specifically, in an embodiment of the present application, an output calculation result of PID regulation is acquired according to a second formula which is specifically as follows:

$$y = {K}_{p} [ ( b \cdot {v}_{t} - {v}_{0} ) + \frac{1}{{T}_{I} \cdot s} ( {v}_{t} - {v}_{0} ) + \frac{{T}_{D} \cdot s}{a \cdot {T}_{D} \cdot s + 1} ( c \cdot {v}_{t} - {v}_{0} ) ] \qquad ( 2 )$$

where, y represents the output calculation result of PID regulation (i.e. a frequency set value), Kₚ represents a proportional gain, s represents a Laplace operator, b represents a proportional action weight, vₜ represents the target speed (a set value), v₀ represents a current speed (a process value), T_{I} represents an integral action time, T_{D} represents a differential action time, a represents a differential delay coefficient (differential delay T_{I}=a×T_{D}), and c represents a differential action weight.

In an optional embodiment of the present application, after determining the target speed for material distribution at each material distribution point according to the total amount of materials, the method further comprises: calculating the advanced announcement time of the target speed according to the target speed, the distance in the fermentation tank and the closed-loop regulation lag time when the average material distribution amount is equal to the target average material distribution amount; and

performing a closed-loop control to the total amount of materials based on the advanced announcement time of the target speed.

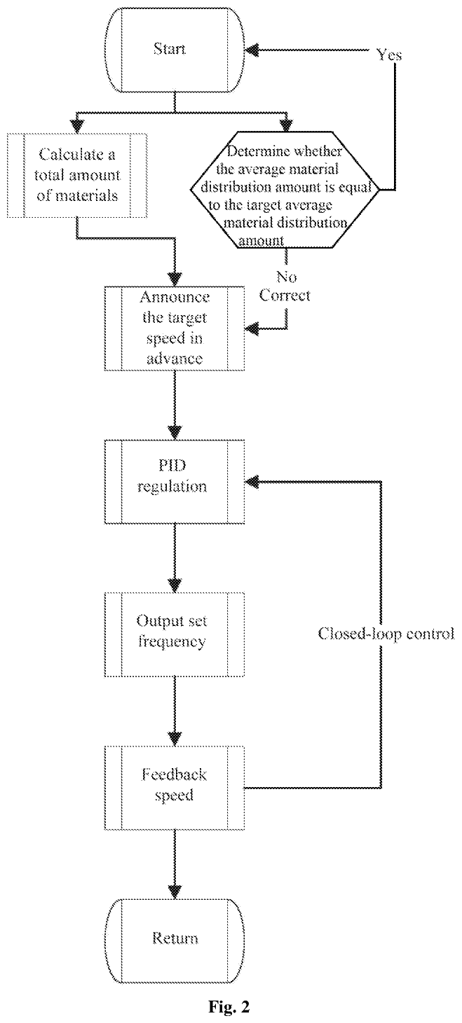

As shown in FIG. 2, a flow chart of uniform material distribution control using the above method is provided.

The flow chart is mainly divided into three parts: material calculation, target speed calculation and announcement, and PID closed-loop control.

During material calculation, a total amount of materials and a cumulative material distribution amount and an average material distribution amount are calculated.

During target speed calculation and announcement, a corresponding target speed is calculated according to the total amount of materials, the target speed is corrected according to the average material distribution amount and a target average material distribution amount, and an advanced announcement time of the target speed is calculated according to a transmission speed, a position of the apparatus, and a closed-loop regulation lag time.

During PID closed-loop control, a PID closed-loop regulation is performed according to the target speed.

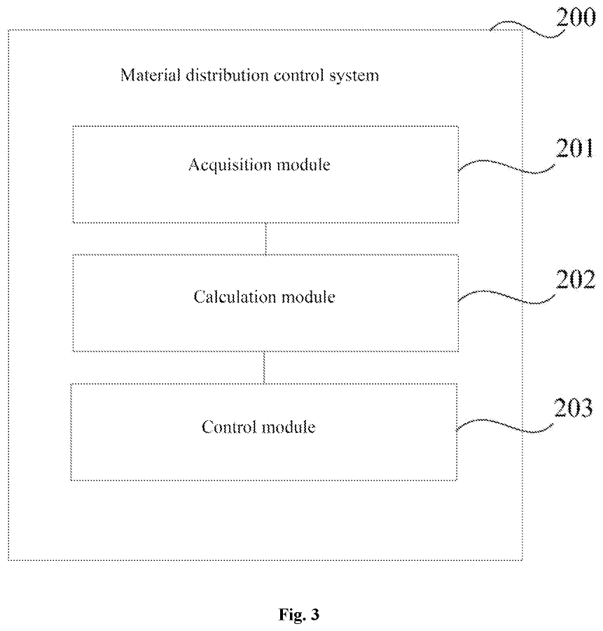

Referring to FIG. 3, a block diagram of a uniform material distribution control system 200 for fermentation tank provided in an embodiment of the present application is shown. As shown in FIG. 3, the system 200 may comprise: an acquisition module 201, a calculation module 202, and a control module 203.

The acquisition module 201 is configured to acquire a total amount of materials in the fermentation tank and an average material distribution amount at each material distribution point.

The calculation module 202 is configured to determine a target speed for material distribution at each material distribution point according to the total amount of materials, correct the target speed according to the average material distribution amount and a target average material distribution amount when the average material distribution amount is not equal to the target average material distribution amount, and calculate an advanced announcement time of the target speed according to the corrected target speed, a distance in the fermentation tank and a closed-loop regulation lag time.

The control module 203 is configured to perform a closed-loop control to the total amount of materials based on the advanced announcement time of the target speed.

In an optional embodiment of the present application, in the acquisition module, acquiring an average material distribution amount at each material distribution point comprises:

acquiring a cumulative material distribution amount in the fermentation tank, and determining the average material distribution amount at each material distribution point according to a number of material distribution points in the fermentation tank.

In an optional embodiment of the present application, after the target speed for material distribution at each material distribution point is determined according to the total amount of materials, the calculation module is further configured to:

calculate the advanced announcement time of the target speed according to the target speed, the distance in the fermentation tank and the closed-loop regulation lag time when the average material distribution amount is equal to the target average material distribution amount;

and the control module is then configured to perform a closed-loop control to the total amount of materials based on the advanced announcement time of the target speed.

In an optional embodiment of the present application, in the control module, performing a closed-loop control to the total amount of materials based on the advanced announcement time of the target speed comprises:

regulating, by PID, the total amount of materials in the fermentation tank;

acquiring a current feedback speed according to an output set frequency of current PID regulation; and

regulating, by PID, the current total amount of materials based on the current feedback speed.

For the specific definitions to the uniform material distribution control system for fermentation tank, reference may be made to the above definitions to the uniform material distribution control method for fermentation tank, which will not be repeated here. The individual modules in the above-mentioned uniform material distribution control system for fermentation tank may be implemented in whole or in part by a software, a hardware and a combination thereof. The above-mentioned individual modules may be embedded in or independent of a processor within a computer equipment in the form of hardware, or may be stored in a memory within the computer equipment in the form of software, such that the processor may call and execute corresponding operations of the above individual modules.

The various technical features of the above-mentioned embodiments may be combined arbitrarily. For sake of simplicity of the description, not all of the possible combinations of the various technical features in the above-mentioned embodiments are described. However, as long as the combinations of these technical features are not contradictory, they should be considered as falling into the scope recited in this description.

The above-mentioned embodiments only specially illustrate several implementations of the present application in detail, but are not regarded as limitations to the present application. It should be noted that, those skilled in the art may make various modifications and improvements without departing from the concept of the present application, all of which fall into the scope of protection of the present application. Therefore, the scope of protection of the present application shall be subject to the scope defined in the attached claims.

1. A uniform material distribution control method for fermentation tank, characterized in that, the method comprises:
acquiring a total amount of materials in the fermentation tank and an average material distribution amount at each material distribution point;
determining a target speed for material distribution at each material distribution point according to the total amount of materials, wherein the target speed is corrected according to the average material distribution amount and a target average material distribution amount when the average material distribution amount is not equal to the target average material distribution amount, and an advanced announcement time of the target speed is calculated according to the corrected target speed, a distance in the fermentation tank and a closed-loop regulation lag time; and
performing a closed-loop control to the total amount of materials based on the advanced announcement time of the target speed,
wherein, after determining the target speed for material distribution at each material distribution point according to the total amount of materials, the method further comprises: calculating the advanced announcement time of the target speed according to the target speed, the distance in the fermentation tank and the closed-loop regulation lag time when the average material distribution amount is equal to the target average material distribution amount; and performing a closed-loop control to the total amount of materials based on the advanced announcement time of the target speed, and wherein,
calculating the advanced announcement time of the target speed according to the target speed, the distance in the fermentation tank and the closed-loop regulation lag time comprises:
determining the regulation lag time Δt and the target speed vₜ according to a first formula which is specifically as follows:
$$Δ t = {k}_{t} \frac{l \frac{{w}_{0}}{w} - s}{{v}_{0}} , {v}_{t} = \frac{l}{\frac{w}{{f}_{t}} - Δ t}$$
where, kₜ represents a compensation coefficient, s represents a current position, l represents a total length, w represents a target material distribution amount, w₀ represents a current material distribution amount, v₀ represents a current speed, and fₜ represents an instantaneous material flow rate.

2. The method according to claim 1, characterized in that, acquiring an average material distribution amount at each material distribution point comprises:
acquiring a cumulative material distribution amount in the fermentation tank, and determining the average material distribution amount at each material distribution point according to a number of material distribution points in the fermentation tank.

3. The method according to claim 1, characterized in that, performing a closed-loop control to the total amount of materials based on the advanced announcement time of the target speed comprises:
regulating, by PID, the total amount of materials in the fermentation tank;
acquiring a current feedback speed according to an output set frequency of current PID regulation; and
regulating, by PID, the current total amount of materials based on the current feedback speed.

4. The method according to claim 3, characterized in that, an output calculation result of PID regulation is acquired according to a second formula which is specifically as follows:
$$y = {K}_{p} [ ( b \cdot {v}_{t} - {v}_{0} ) + \frac{1}{{T}_{I} \cdot s} ( {v}_{t} - {v}_{0} ) + \frac{{T}_{D} \cdot s}{a \cdot {T}_{D} \cdot s + 1} ( c \cdot {v}_{t} - {v}_{0} ) ]$$
where, y represents the output calculation result of PID regulation, Kₚ represents a proportional gain, s represents a Laplace operator, b represents a proportional action weight, vₜ represents the target speed, v₀ represents a current speed, T_{I} represents an integral action time, T_{D} represents a differential action time, a represents a differential delay coefficient, and c represents a differential action weight.

5. A uniform material distribution control system for fermentation tank, characterized by comprising:
an acquisition module, configured to acquire a total amount of materials in the fermentation tank and an average material distribution amount at each material distribution point;
a calculation module, configured to determine a target speed for material distribution at each material distribution point according to the total amount of materials, correct the target speed according to the average material distribution amount and a target average material distribution amount when the average material distribution amount is not equal to the target average material distribution amount, and calculate an advanced announcement time of the target speed according to the corrected target speed, a distance in the fermentation tank and a closed-loop regulation lag time; and
a control module, configured to perform a closed-loop control to material distribution based on the advanced announcement time of the target speed,
wherein, the calculation module is further configured to calculate the advanced announcement time of the target speed according to the target speed, the distance in the fermentation tank and the closed-loop regulation lag time when the average material distribution amount is equal to the target average material distribution amount;
the control module is further configured to perform a closed-loop control to the total amount of materials based on the advanced announcement time of the target speed; and wherein,
calculating the advanced announcement time of the target speed according to the target speed, the distance in the fermentation tank and the closed-loop regulation lag time comprises:
determining the regulation lag time Δt and the target speed vₜ according to a first formula which is specifically as follows:
$$Δ t = {k}_{t} \frac{l \frac{{w}_{0}}{w} - s}{{v}_{0}} , {v}_{t} = \frac{l}{\frac{w}{{f}_{t}} - Δ t}$$
where, kₜ represents a compensation coefficient, s represents a current position, l represents a total length, w represents a target material distribution amount, w₀ represents a current material distribution amount, v₀ represents a current speed, and fₜ represents an instantaneous material flow rate.

6. The system according to claim 5, characterized in that, in the acquisition module, acquiring an average material distribution amount at each material distribution point comprises:
acquiring a cumulative material distribution amount in the fermentation tank, and determining the average material distribution amount at each material distribution point according to a number of material distribution points in the fermentation tank.

7. The system according to claim 5, characterized in that, in the control module, performing a closed-loop control to the total amount of materials based on the advanced announcement time of the target speed comprises:
regulating, by PID, the total amount of materials in the fermentation tank;
acquiring a current feedback speed according to an output set frequency of current PID regulation; and
regulating, by PID, the current total amount of materials based on the current feedback speed.
